(19) United States
(12) Patent Application Publication
(10) Pub. No.: US 2025/0152077 A1
(43) Pub. Date: May 15, 2025

(54) METHOD FOR PROVIDING DIGITAL FEEDBACK FOR ACTIVATION OR INHIBITION OF TARGET BRAIN REGIONS

(71) Applicant: ExeBrain Co., Ltd., Taipei City (TW)

(72) Inventors: Shu-Chun Kuan, Taipei City (TW); Jason Chun-Cheng Lin, Taipei City (TW)

(21) Appl. No.: 18/829,409

(22) Filed: Sep. 10, 2024

(30) Foreign Application Priority Data
Nov. 9, 2023 (TW) 112143314

(51) Int. Cl. A61B 5/375; G09B 19/00

(52) U.S. Cl. CPC A61B 5/375; G09B 19/00; A61B 2505/09

(57) ABSTRACT

A method for providing digital feedback for activation or inhibition of target brain regions is disclosed. The method involves utilizing a digital interface for conducting physiological feedback training based on physiological signals. A training configuration is executed according to the physiological feedback training. An execution result is generated, including physiological signals and behavioral data. The execution result is analyzed, and if the result meets the expected outcome, the training configuration is maintained. If the execution result does not meet the expected outcome, another training configuration is selected, and the alternative training configuration is executed. This approach allows real-time activation or inhibition specifically targeted at the desired brain regions, avoiding unnecessary activation or inhibition of non-targeted brain regions, thereby reducing the time and number of training sessions required to achieve the intended goals.

This application claims the priority of Taiwanese patent application No. 112143314, filed on Nov. 9, 2023, which is incorporated herewith by reference.

### PRIOR ART

Prior art biofeedback training mainly uses wireless devices at the input terminal, for example, using a pair of electrode patches to compare brainwave changes before and after training in three areas of the parietal lobe, using a pair of electrode patches to detect the impact of neurophysiological feedback on sensorimotor rhythm (SMR), or collecting physiological signals and upload physiological data to the cloud platform for analysis through wired or wireless transmission modules. Individual users need to open the APP or related applications to retrospectively read the physiological device during sleep periods. However, prior art technology usually prevents subjects from immediately obtaining physiologically relevant information such as brainwaves or heartbeat variations, and they need to wait for hours to days for interpretation.

At the same time, although the prior art intelligent bed health management system also collects physiological signals, what is collected is the physiological signals of individuals while sleeping in bed. Physiological signals are uploaded to the cloud platform for analysis through wired or wireless transmission modules. The individual needs to open the relevant application to retrospectively read the physiological device during sleep. The disadvantage is that individual physiological signals cannot be immediately processed and fed back to the subject after being transmitted.

In addition, although there is a neurofeedback mechanism for functional magnetic resonance imaging (fMRI), the equipment for magnetic resonance imaging is quite expensive and is mostly installed in medical institutions. The signal collection to imaging process takes more than 30 minutes, and the calculation of the feedback mechanism also takes more than 10 minutes. It is impossible to achieve remote configuration and real-time (within 1 minute) analysis feedback.

Please refer to the left half of FIG. 7, in the current method of brain area or brain wave training for subjects, by comparing the brain area or brain wave results to be trained, sound and light feedback is given after the expected goal is achieved, which analyzes the entire brain area or brain waves for training. However, it is not possible to perform activation or inhibition training on a specific brain area, so the time and number of training required are very lengthy.

### BACKGROUND OF THE INVENTION

The present invention relates to the technical field of neurofeedback, in particular to a method for providing digital feedback for activation or inhibition of target brain regions.

### SUMMARY OF THE INVENTION

A primary objective of the present invention is to provide a method for providing digital feedback for activation or inhibition of target brain regions. Provides digital (gamification) feedback of activation or inhibition for target brain areas, using neurophysiological signals and physiological feedback training. The digital training therapy remote application performs digital prescription, digital therapy, games (i.e. visual) and music (i.e. auditory) and NFB (Neurofeedback, neurofeedback) interaction to activate/inhibit brain area network operations, so as to achieve the effect of immediate and targeted activation or inhibition of the target brain area. It avoids unnecessary activation or inhibition of other non-target brain areas, thereby reducing the time and number of training required to achieve the expected goal.

A method for providing digital feedback for activation or inhibition of target brain regions is disclosed. The method comprises Step SA: performing physiological feedback training based on a physiological signal through a digital interface; Step SB: executing a training configuration according to the physiological feedback training; Step SC: outputting an execution result, wherein the execution includes a physiological signal and a behavioral data; and Step SD: analyzing whether the execution result is as expected, if the execution result is as expected, maintain the training configuration; if the execution result is not as expected, change to another training configuration and execute the other training configuration.

In some embodiments, the physiological feedback training includes a game training.

In some embodiments, the training configuration includes attention, perceptual processing, visual space, language semantics, working memory, logical reasoning, emotional stimulation and social cognition.

In some embodiments, the execution result includes a signal result, a physical and mental status, and a performance index.

In some embodiments, the method further comprises using a brainwave collection device to capture the physiological signal of a subject before the step SA, wherein the physiological signal is a channel brainwave data (e.g. 19-channel brainwave data) captured from different brain areas of the subject.

In some embodiments, the digital interface is a mobile communication device.

In some embodiments, the physiological signal includes a brainwave amplitude, a brainwave frequency, a brain region location and a pattern characteristic.

In some embodiments, the method further comprises returning to step SB for multiple cycles after the step SD.

### BRIEF DESCRIPTION OF DRAWINGS

Aspects of the present invention are best understood from the following detailed description when read with the accompanying figures. It is noted that, in accordance with the standard practice in the industry, various features are not drawn to scale. In fact, the dimensions of the various features may be increased or reduced for clarity of discussion.

FIG. 1 is a flowchart of a method for providing digital feedback for activation or inhibition of target brain regions in accordance with embodiments of the present invention.

FIG. 2 is a schematic view of a brainwave collection device used in the method for providing digital feedback for activation or inhibition of target brain regions in accordance with embodiments of the present invention.

FIG. 3 is a side view of a brainwave collection device used in the method for providing digital feedback for activation or inhibition of target brain regions in accordance with embodiments of the present invention.

FIG. 4 is a schematic view of brainwave feature analysis of the method for providing digital feedback for activation or inhibition of target brain regions in accordance with embodiments of the present invention.

FIG. 5 is a schematic diagram of the usage state of the method for providing digital feedback for activation or inhibition of target brain regions in accordance with embodiments of the present invention.

FIG. 6 is a schematic diagram of the method for providing digital feedback for activation or inhibition of target brain regions in accordance with embodiments of the present invention.

FIG. 7 is a schematic diagram of the method for providing digital feedback for activation or inhibition of target brain regions in accordance with embodiments of the present invention, which comparing the present method with conventional techniques.

FIG. 8 is a schematic diagram illustrating the method for providing digital feedback for activation or inhibition of target brain regions in a game in accordance with embodiments of the present invention.

FIG. 9 is a schematic diagram illustrating the method for providing digital feedback for activation or inhibition of target brain regions in progress in a game in accordance with embodiments of the present invention.

FIG. 10 is a schematic diagram illustrating the method for providing digital feedback for activation or inhibition of target brain regions to produce results in a game in accordance with embodiments of the present invention.

FIG. 11 is a schematic diagram of a feedback interface of the method for providing digital feedback for activation or inhibition of target brain regions in accordance with embodiments of the present invention.

### DETAILED DESCRIPTION OF THE INVENTION

It will be appreciated that, although specific embodiments of the present invention are described herein for purposes of illustration, various modifications may be made without departing from the spirit and scope of the present invention.

In the following description, certain specific details are set forth in order to provide a thorough understanding of various aspects of the disclosed subject matter. However, the disclosed subject matter may be practiced without these specific details. In some instances, well-known structures and methods of power delivery comprising embodiments of the subject matter disclosed herein have not been described in detail to avoid obscuring the descriptions of other aspects of the present invention.

Unless the context requires otherwise, throughout the specification and claims that follow, the word “comprise,” “have,” “include,” and variations thereof, such as “comprises,” “comprising,” “having,” “including” are to be construed in an open, inclusive sense, that is, as “including, but not limited to.”

Reference throughout the specification to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same aspect. Furthermore, the particular features, structures, or characteristics may be combined in any suitable manner in one or more aspects of the present invention.

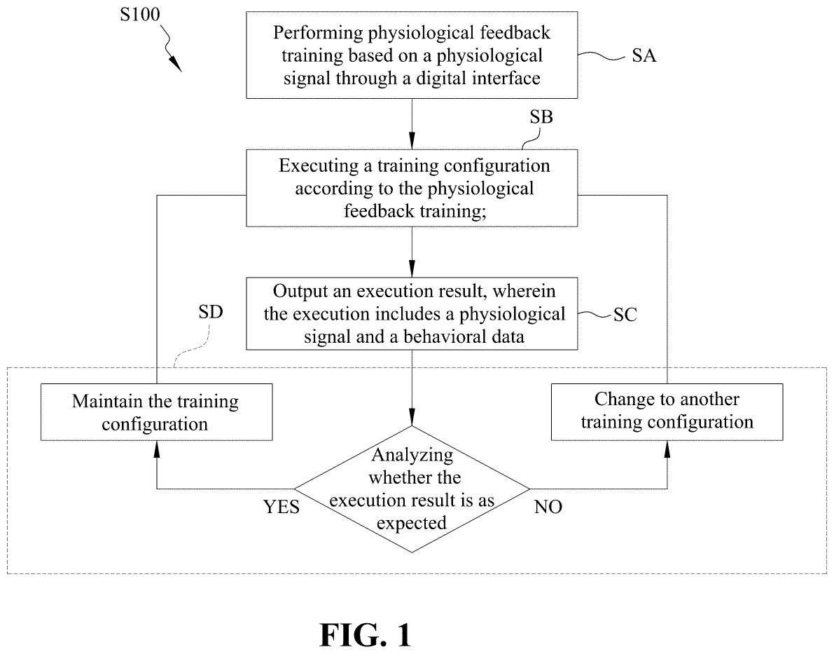

FIG. 1 is a flowchart of a method for providing digital feedback for activation or inhibition of target brain regions in accordance with embodiments of the present invention. Please refer to FIG. 1, the method S100 for providing digital feedback for activation or inhibition of target brain regions includes Step SA: performing physiological feedback training based on a physiological signal through a digital interface; Step SB: executing a training configuration according to the physiological feedback training; Step SC: outputting an execution result, wherein the execution includes a physiological signal and a behavioral data; and Step SD: analyzing whether the execution result is as expected, if the execution result is as expected, maintain the training configuration; if the execution result is not as expected, change to another training configuration and execute the other training configuration.

In some embodiments, the method S100 for providing digital feedback for activation or inhibition of target brain regions further comprises returning to step SB for multiple cycles after the step SD to execute multiple training configurations or obtain the best training configuration immediately and in a short time.

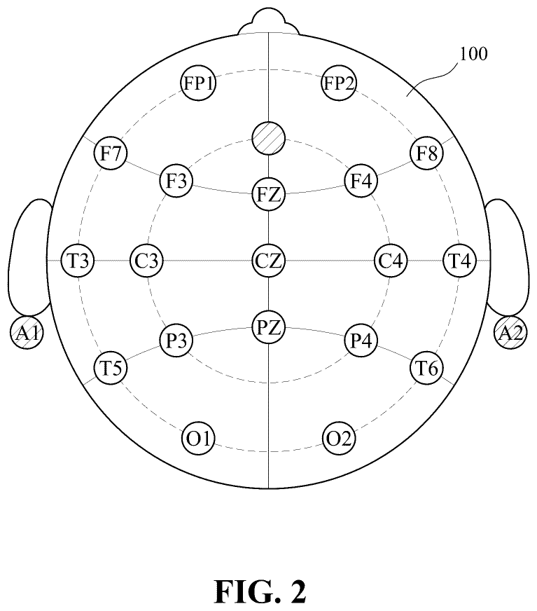

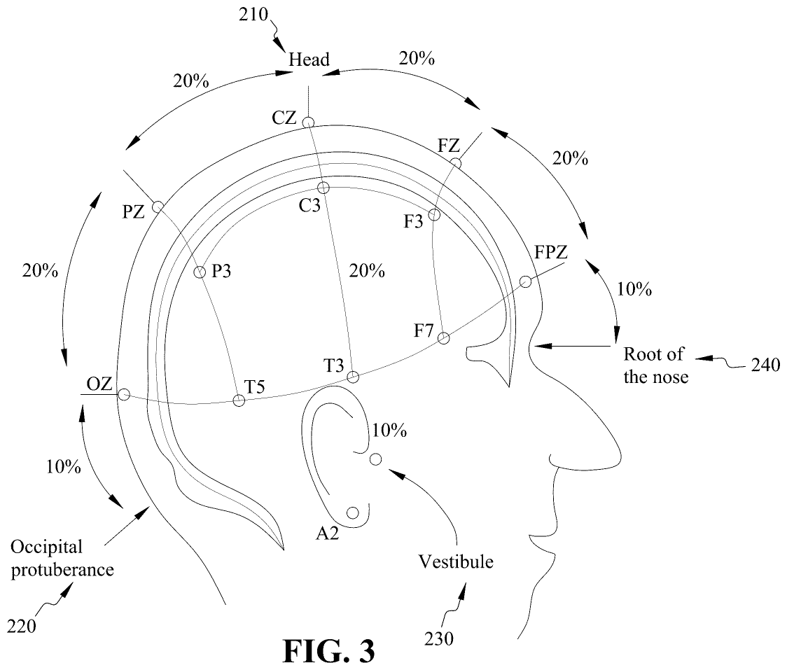

In some embodiments, the method for providing digital feedback for activation or inhibition of target brain regions further comprises using a brainwave collection device 100 to capture the physiological signal of a subject before the step SA, wherein the physiological signal is a channel brainwave data (e.g. 19-channel brainwave data) captured from different brain areas of the subject. It will be described in detail in FIG. 2 and FIG. 3. The brainwave collection device 100 can be set up in the subject's home or in a public place such as a medical unit

In some embodiments, the digital interface 300 may be a mobile communication device, such as a mobile phone, but is not limited thereto.

FIG. 2 is a schematic view of a brainwave collection device used in the method for providing digital feedback for activation or inhibition of target brain regions in accordance with embodiments of the present invention. FIG. 3 is a side view of a brainwave collection device used in the method for providing digital feedback for activation or inhibition of target brain regions in accordance with embodiments of the present invention.

Please refer to FIGS. 2 and 3, the brainwave collection device 100 is worn in contact with the subject's head 210, occipital protuberance 220, vestibule 230 and the root of the nose 240, and the brainwave collection device 100 includes electrodes C3, C4, P3, P4, O1, O2, FP1, FP2, FZ, CZ, PZ, T3, T4, T5, T6, F3, F4, F7, F8 and ear electrodes A1 and A2, that is, the brain activity areas are positioned through the combination of brainwave sites and brainwave patterns of the 19 channels of the above 19 electrodes.

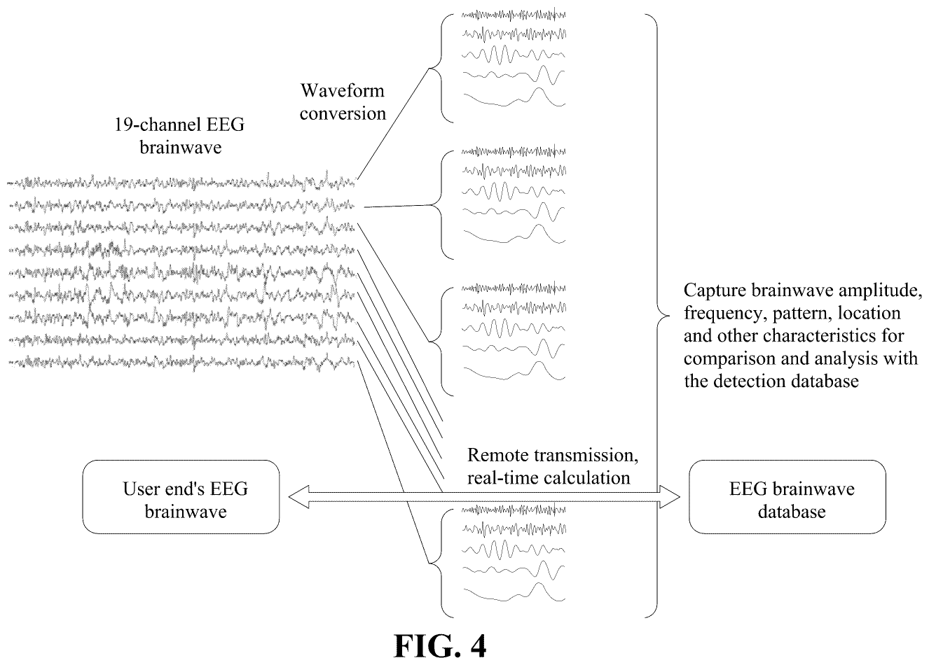

FIG. 4 is a schematic view of brainwave feature analysis of the method for providing digital feedback for activation or inhibition of target brain regions in accordance with embodiments of the present invention. In some embodiments, brain activity areas are positioned based on the combination of 19-channel brainwave sites and brainwave patterns of the brainwave collection device used above. Therefore, the biological data may be a 19-channel EGG (Electroencephalography) brainwave data. Therefore, it is recommended that the training parameters mainly use the 19-channel brainwave data, convert it into a standard score according to the calculation, and then present an order list according to the deviation mean. The brainwave data may include amplitude, frequency, site and pattern characteristics, but is not limited thereto. As shown in FIG. 4, The brainwave characteristics detected through the 19 channels (that is, the 19 electrodes FP1, FP2, F3, F4, F7, F8, FZ, T3, C3, CZ, C4, T4, T5, P3, PZ, P4, T6, O1, O2 in FIGS. 2 and 3) of the brainwave collection device 100 include four characteristic parameters: vibration, frequency, brainwave site (sites of 19 electrodes FP1, FP2, F3, F4, F7, F8, FZ, T3, C3, CZ, C4, T4, T5, P3, PZ, P4, T6, O1, O2), and amplitude, frequency, shape, and position of the brainwave pattern. The four characteristic parameters constitute the brainwave database of different ethnic groups through. The sites of the 19 channels are basically positioning to predict brain activity areas backwards through brainwave characteristics, which can be used as parameters for the above comparison and training. In this embodiment, the brainwaves collected by the brainwave collection device 100 may be compared with the brainwave patterns to obtain a basic score. As shown in FIG. 4, after comparison and analysis with the brainwave database 400, a benchmark point for the index score may be generated. This benchmark point is also the difference compared to the norm, in similar groups (same age, same education level, same gender, etc.).

For example, if the brainwave collected by a subject through the brainwave collection device 100 is converted into a score of X, but the ideal score of the subject compared with the database should be Y, then during the neurofeedback training process, the goal is to reduce the difference between X and Y, and the subject will receive feedback messages when the difference is reduced to a certain ratio. After the subject accepts the feedback message, he or she can perform another brainwave pattern comparison. At this time, the brainwaves collected by the brainwave collection device 100 can be converted into a new benchmark score X′. This X′ will also be compared with the database, and the new neurofeedback training goal is to shorten the difference between X′ and Y. Neurofeedback training is much like muscle training for the brain. If you want to exercise a certain muscle (biceps of the hand, six-pack muscles of the abdomen, thigh muscles of the legs, etc.), the muscle endurance before exercise is X, but the goal is to obtain Y strength, then you will gradually train specific muscle groups until X−Y gets closer and closer. For example, if you want to be able to lift a 30 kg (Y) dumbbell, but currently the user end can only lift 10 kg (X), if the user end can lift 15 kg, then feedback will be given (X−Y is shortened by a certain percentage). After exercising for a period of time and reassessing, the user end can lift a 20 kg (X′) dumbbell. At this time, the user end may need to lift a 25 kg dumbbell (X′−Y is shortened by a certain proportion) to get feedback. For example, if a subject with inattention wants to improve his concentration through brain training, he can analyze the collected brainwaves. If it is found that the frontal lobe area of the brain is overactivated compared with the norm, feedback can be used to gradually reduce the difference between X and Y.

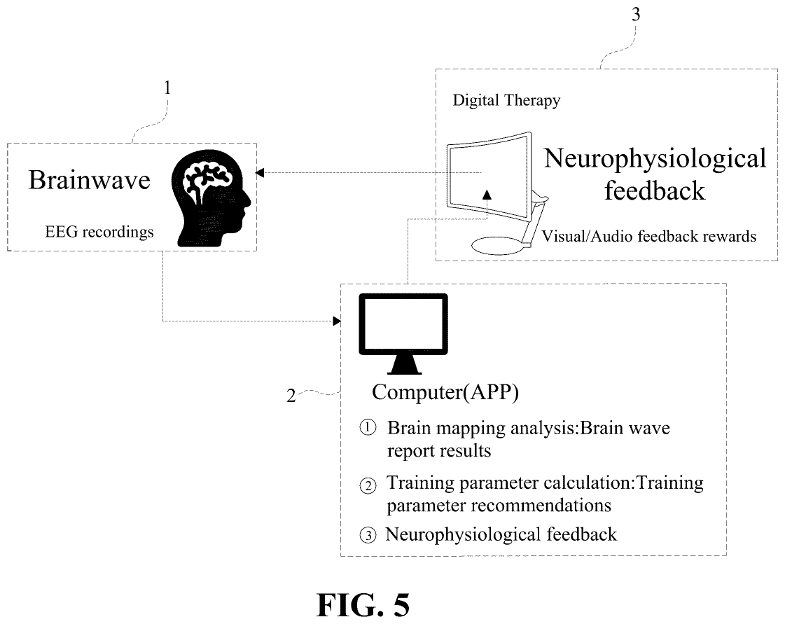

FIG. 5 is a schematic diagram of the usage state of the method for providing digital feedback for activation or inhibition of target brain regions in accordance with embodiments of the present invention. Please refer to FIG. 5, in step A, real-time recording and real-time analysis of the subjects are performed (i.e., block 1); the physiological signal is then provided to an application software (i.e., block 2) stored in a storage memory medium (such as a computer), and the application software analyzes the brain map and provides a report result; then perform training parameter calculation and provide training parameter suggestions (i.e. training configuration); afterwards, digital therapy is carried out according to the training parameter recommendations (i.e. block 3), which means that neurophysiological feedback is generated, and then visual and visual sound and light effects will appear when the brainwaves are in the best state.

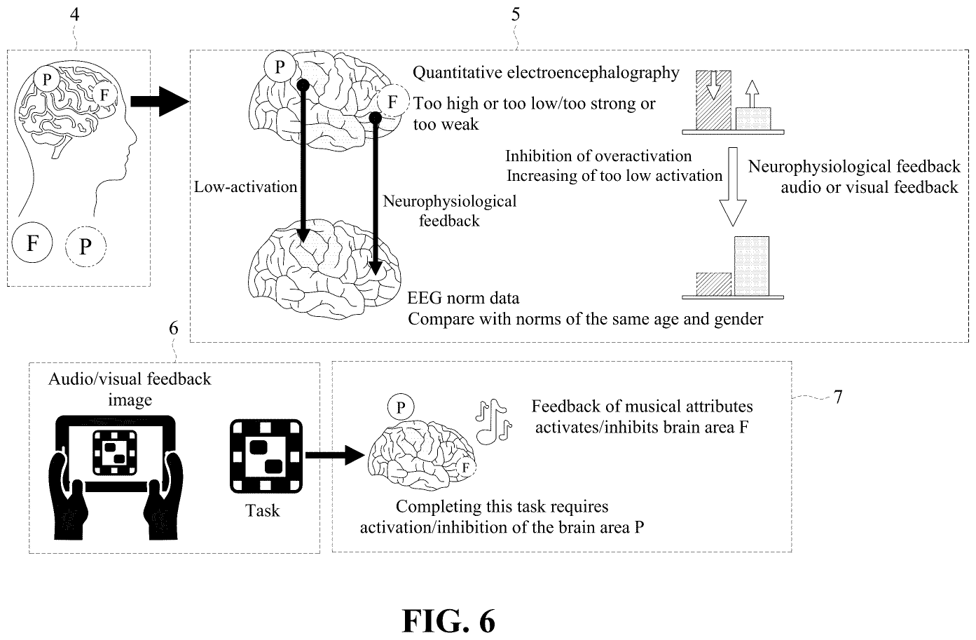

FIG. 6 is a schematic diagram of the method for providing digital feedback for activation or inhibition of target brain regions in accordance with embodiments of the present invention. Please refer to FIG. 5 and FIG. 6 at the same time to perform training on the subject's brain area P and brain area F (i.e. block 4), after obtaining the EEG results (that is, the brainwaves in the target brain area are too high or too low/too strong or too low) and the EEG norm data (that is, comparing the norms with the same age and gender), confirm that the target brain area P of the subject is under-activated, while the target brain area F is over-activated. Therefore, through sound/visual feedback images, such as a game task (i.e., physiological feedback training) (i.e., block 6), the target brain area P is activated and the target brain area F is inhibited by feedbacking the music attributes. (i.e. Box 7), thereby achieving activation or inhibition of target brain areas.

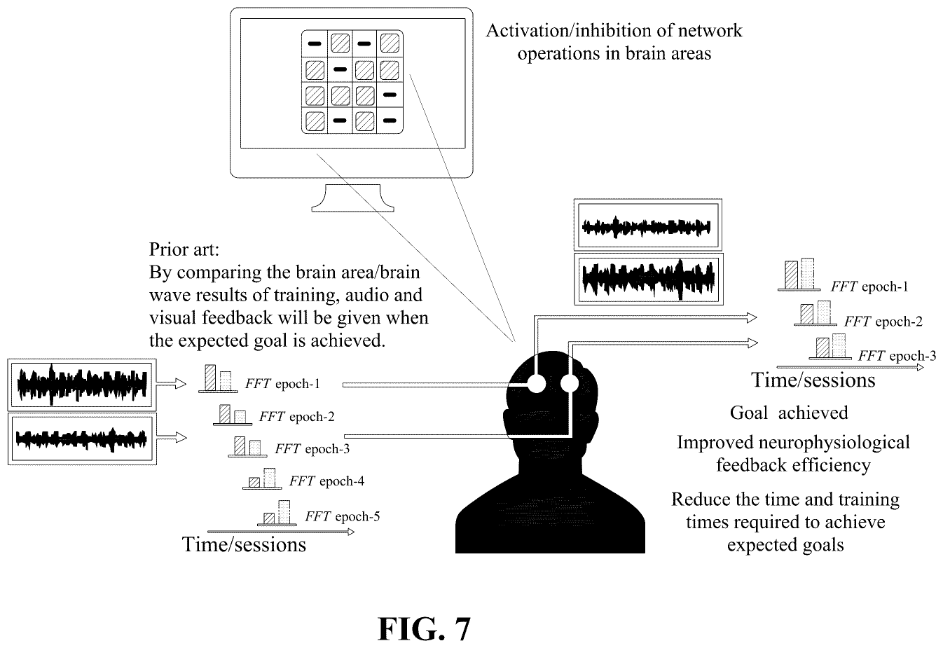

FIG. 7 is a schematic diagram of the method for providing digital feedback for activation or inhibition of target brain regions in accordance with embodiments of the present invention, which comparing the present method with conventional techniques. Please refer to the right half of FIG. 7, after activating/inhibiting the target brain area through training with the aforementioned training configuration, the neurophysiological feedback efficiency of the target brain area of the subject can be improved, and the time and number of training required to achieve the expected goal can be reduced.

FIG. 7 mainly describes the method of “prior art”, that is, through real-time brain wave analysis, after comparing the target area or brainwave frequency band, visual or sound sensory feedback is given when the set target level is reached. However, visual feedback played a more passive role in previous technologies, and sensory feedback can be obtained as long as the set target threshold is reached. However, the technology of the present invention is the feedback of the sensory interface itself, which can also activate or inhibit specific brain areas. For example, we need to train brain area A, and if the brainwave activity characteristics of brain area A (FIG. 4), the game screen will appear (Take a racing car as an example. If the brainwaves match the target characteristic value, the car will continue to run in the screen. If it does not meet the characteristic value, the car will stay still in the screen, just like using brainwaves to control a racing game.) Whether the car moved in the prior art only fed back to the user's current brain wave characteristics, thereby giving back to the user's current status. The racing game itself is only for feedback, but it only plays the role of “passive feedback” in brain training. However, in the present invention, the feedback image itself plays the role of “active feedback” that activates or inhibits specific brain areas. For example, if the racing feedback image is changed to airplane driving, although the feedback image is all about moving vehicles, the airplane can also activate/inhibit the A brain area.

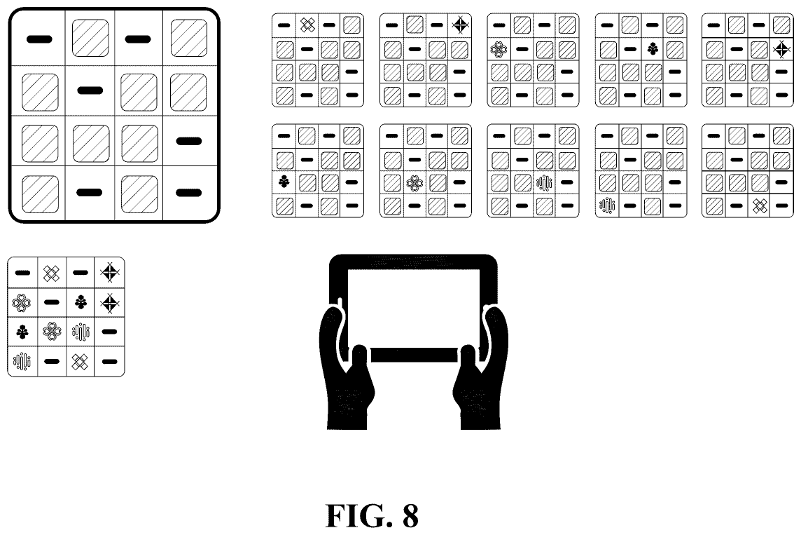

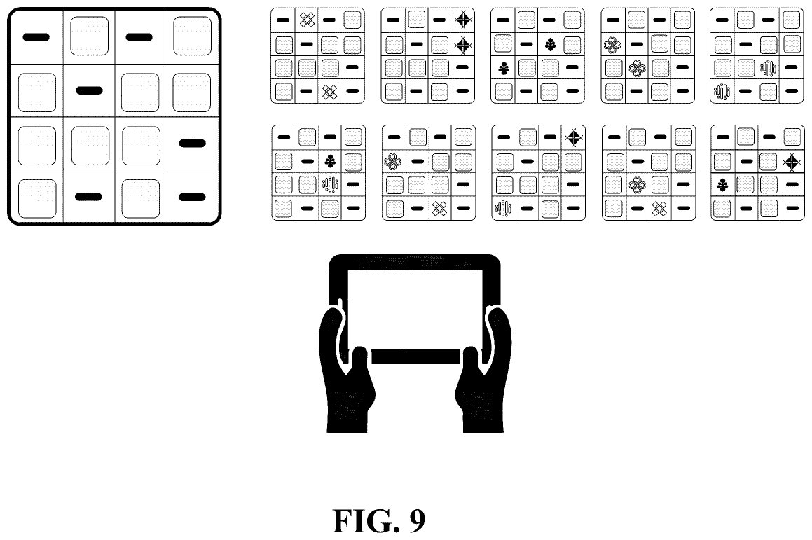

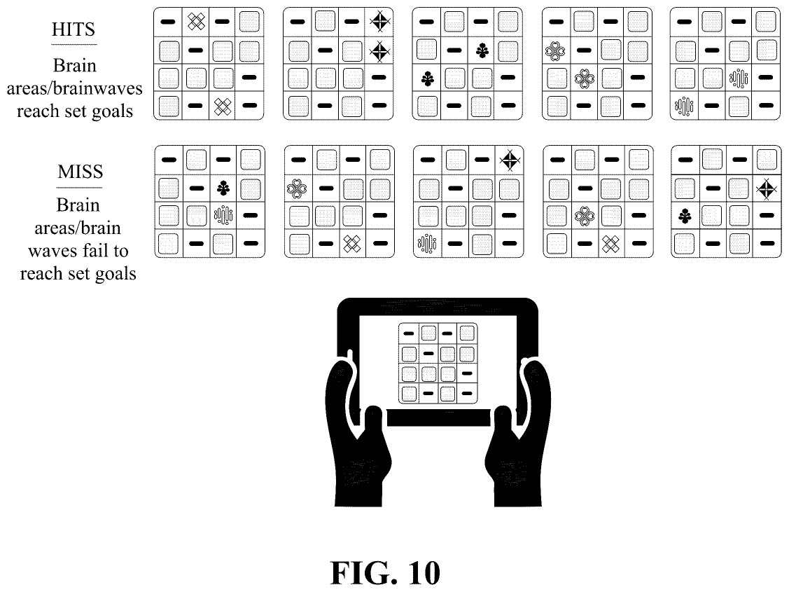

FIG. 8 is a schematic diagram illustrating the method for providing digital feedback for activation or inhibition of target brain regions in a game in accordance with embodiments of the present invention. FIG. 9 is a schematic diagram illustrating the method for providing digital feedback for activation or inhibition of target brain regions in progress in a game in accordance with embodiments of the present invention. FIG. 10 is a schematic diagram illustrating the method for providing digital feedback for activation or inhibition of target brain regions to produce results in a game in accordance with embodiments of the present invention.

Combined with FIGS. 7, 8 and 9, for example, the game diagram of FIGS. 8 and 9: the technology of prior art may perform card flipping actions as long as the brainwaves matched the characteristics to be trained, that is, visual feedback was to use brain waves to flip cards (similar to the above-mentioned use of brain waves to drive a racing car); the current technology is that brain waves match the training characteristics and can be used to flip cards, however, these cards are also followed by cognitive-related tasks to activate or inhibit specific brain areas (similar to the above-mentioned use of brainwaves to fly a plane). Although driving a car and driving an airplane are both driving, the former is in a 2D plane and the latter is in a 3D space.

Please refer to FIGS. 8 to 10, which provide a card flipping game for memorizing a pair of cards of the same suit in different positions as a homework task for training. During neurofeedback, the brain area/brainwave results to be trained are determined. When the desired goal is met (reaching a predetermined threshold), the card may be revealed. At this time, the subject needs to remember the relative position of each card; this task is related to the brain area of memory processing, and performing this task alone may train the memory surface. However, performing this task through neurofeedback signals may enhance more memory surfaces (i.e., activate the target brain areas of memory) and consolidate the enhancement of neurophysiological feedback to the brain.

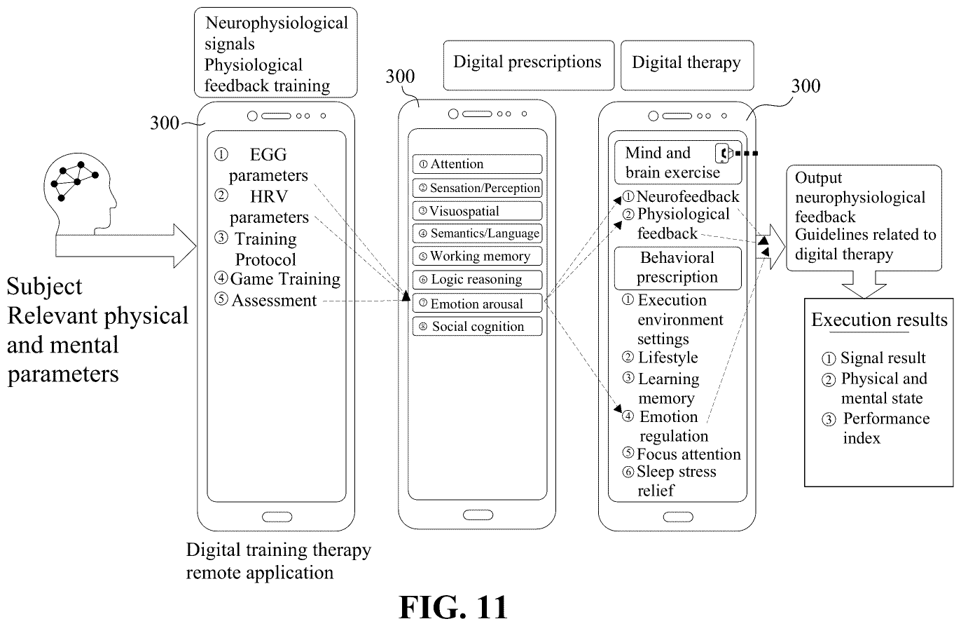

FIG. 11 is a schematic diagram of a feedback interface of the method for providing digital feedback for activation or inhibition of target brain regions in accordance with embodiments of the present invention. Please refer to FIG. 11, the present invention may be implemented by an application program on a mobile phone. The physiological signals captured from the subject are remotely transmitted to the mobile phone (i.e., the digital interface 300) via the Internet (step SA); and perform neurophysiological signal physiological feedback training through the application (i.e. APP), that is, enter a game training configuration (step SB); after entering the game training configuration, the training of the game training configuration is executed and an execution result is generated (step SC), wherein the training includes digital prescription and digital training therapy, the scope of digital prescription at least includes attention, perceptual processing, visual space, language semantics, working memory, logical reasoning, emotional stimulation and social cognition, the digital training treatment includes mind and brain training and behavioral prescriptions (including relevant guidelines), wherein mind and brain training includes neurofeedback and physiological feedback, and the behavioral prescriptions include execution environment settings, lifestyle, learning and memory, emotion regulation, concentration and attention, and sleep stress relief. The execution result may include a signal result, a physical and mental status, and a performance index.

Please refer to the dotted line indication in FIG. 11. For example, the physiological signals provided in the neurophysiological signal physiological feedback training include EEG (Electroencephalography) parameters, HRV (Heart Rate Variability Analysis, heart rate variability) parameters and evaluation values. After that, it enters the “emotional stimulation” training configuration, and then provides neurofeedback, physiological feedback (Digital Prescriptions) and emotional regulation (Behavioral Prescriptions (Digital Therapy)), and finally outputs the execution results. The training configuration may be further adjusted or cycled according to the aforementioned flowchart in FIG. 1.

This invention emphasizes the digitalized method, that is, the target brain area may be trained without using brainwaves. But if there are brainwaves, the effect will be 1+1>2, that is to say, including physiological feedback through physiological signals to optimize the brain, and digital game feedback itself (only the game itself, not the physiological signals) may also optimize the brain. The effect of physiological feedback training combined with digital feedback methods will be doubled. Part of digitalized feedback is to calculate relevant brain activation areas through the user's behavioral responses or behavioral characteristics (such as response accuracy, reaction time, optimal performance level, etc.), but not through the physiological signals themselves. The principle behind this is that behavioral characteristics reflect brain activity. At present, this invention may also calculate the characteristics of high activation/low activation of the brain through behavioral characteristics, and improve behavioral characteristics through digitalized games to enhance brain function, but the process itself does not include the analysis of physiological signals.

In summary, the method S100 provided by the present invention for providing digital feedback of activation or inhibition for target brain areas, provides digital (gamification) feedback of activation or inhibition for target brain areas, using neurophysiological signals and physiological feedback training. The digital training therapy remote application performs digital prescription, digital therapy, games (i.e. visual) and music (i.e. auditory) and NFB (Neurofeedback, neurofeedback) interaction to activate/inhibit brain area network operations, so as to achieve the effect of immediate and targeted activation or inhibition of the target brain area. It avoids unnecessary activation or inhibition of other non-target brain areas, thereby reducing the time and number of training required to achieve the expected goal.

1. A method for providing digital feedback for activation or inhibition of target brain regions, comprising:
Step SA: performing physiological feedback training based on a physiological signal through a digital interface;
Step SB: executing a training configuration according to the physiological feedback training;
Step SC: outputting an execution result, wherein the execution includes a physiological signal and a behavioral data; and
Step SD: analyzing whether the execution result is as expected, if the execution result is as expected, maintain the training configuration; if the execution result is not as expected, change to another training configuration and execute the other training configuration.

2. The method according to claim 1, wherein the physiological feedback training includes a game training.

3. The method according to claim 1, wherein the training configuration includes attention, perceptual processing, visual space, language semantics, working memory, logical reasoning, emotional stimulation and social cognition.

4. The method according to claim 1, wherein the execution result includes a signal result, a physical and mental status, and a performance index.

5. The method according to claim 1, further comprising using a brainwave collection device to capture the physiological signal of a subject before the step SA, wherein the physiological signal is channel brainwave data captured from different brain areas of the subject.

6. The method according to claim 1, wherein the digital interface is a mobile communication device.

7. The method according to claim 1, wherein the physiological signal includes a brainwave amplitude, a brainwave frequency, a brain region location and a pattern characteristic.

8. The method according to claim 1, further comprising returning to step SB for multiple cycles after the step SD.
